(19) United States
(12) Patent Application Publication
(10) Pub. No.: US 2003/0159857 A1
(43) Pub. Date: Aug. 28, 2003

(54) Electronic scale with body fat and food nutrition/calorie measuring device

(76) Inventors: Bor-Shing Lin, Changhua (TW); Bor-Shyh Lin, Changhua (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/081,858

(22) Filed: Feb. 25, 2002

(51) Int. Cl. G01G 19/22; G01G 19/34; G01G 19/40; A61B 5/04; A61B 10/00; G06F 17/00

(52) U.S. Cl. 177/25.13; 177/25.16; 600/372; 128/921

(57) ABSTRACT

An electronic scale includes a weighing unit coupled to a processing unit. A data memory device is coupled to the processing unit and stores a list of diseases and associated constraints of food consumption. The user may establish personal constitution feature data in the memory and associates the personal constitution feature data with one or more diseases in the disease list. The memory also stores nutrition/calorie data of a number of different categories of foods as a food list. When a particular food is being weighed, the user may select the category of the food in the food list and the processing unit performs calculation of the nutrition/calorie of the particular food. A warning is issued if the user's personal constitution feature data indicates that such an amount of nutrition/calorie is not acceptable to the user for health purposes. Electrodes are coupled to the processing unit for measuring and calculating body fat of the user.

### BACKGROUND OF THE INVENTION

1. Field of the Invention

The present invention relates generally to an electronic scale, and in particular to an electronic scale incorporating a measuring device for user's body fat and nutrition/calorie contained in the food to be consumed by the user for providing health-related warning to the user.

2. Description of the Prior Art

In the modern society, a lot of people are suffering body weight problem. In addition, some people do not take food correctly, causing improper increase of body fat which in turn causes diseases. Thus, controlling food consumption and body weight is of vital importance to a lot of people.

Scales are most commonly used by individuals to control their weights. However, conventional scale can only measure the body weight of a user or food weight. They cannot provide other health related information, such as the user's body fat and the nutrition/calorie contents of the food to be consumed by the user. It is thus desired to provide an electronic scale that is capable to measure the body fat and the nutrition/calorie contents of food to be consumed in order to provide a user with health related information.

### SUMMARY OF THE INVENTION

An object of the present invention is thus to provide an electronic scale having a nutrition/calorie measuring device for measuring the nutrition/calorie contained in the food to be consumed by an individual.

Another object of the present invention is to provide an electronic scale having a body fat measuring device for measuring the body fat of a user.

A further object of the present invention is to provide an electronic scale that is capable to provide correct food consumption information to a user especially for persons having diseases that have constraints to absorption of particular nutrition/calorie.

To achieve the above objects, in accordance with the present invention, there is provided an electronic scale comprising a weighing unit coupled to a processing unit. A memory device is coupled to the processing unit and stores a list of diseases and associated constraints of food consumption. A user may operate the processing unit via a keyboard and accesses the data of the memory. The user may establish personal constitution feature data in the memory and associates the personal constitution feature data with one or more diseases in the disease list. The memory also stores nutrition/calorie data of a number of different categories of foods as a food list. When a particular food is being weighed, the user may select the category of the food in the food list and the processing unit performs calculation of the nutrition/calorie of the particular food. A warning is issued if the user's personal constitution feature data indicates that such an amount of nutrition/calorie is not acceptable to the user for health purposes. Electrodes are coupled to the processing unit. The electrodes are attachable to the user for forming a current loop circuit to measure and calculate body fat of the user in accordance with BIA analysis. The body fat is also stored in the personal constitution feature data.

### BRIEF DESCRIPTION OF THE DRAWINGS

The present invention will be apparent to those skilled in the art by reading the following description of a preferred embodiment thereof, with reference to the attached drawings, in which:

FIG. 1 is a block diagram showing a measuring device constructed in accordance with the present invention;

FIG. 2 is a flow chart showing the measurement of body fat by means of the measuring device in accordance with the present invention;

FIG. 3 is a flow chart showing the establishment and/or modification of personal constitution feature data in the measuring device of the present invention;

FIG. 4 is a flow chart showing the calculation of food nutrition/calorie in accordance with another embodiment of the present invention;

FIG. 5 shows a main menu of the operation of the measuring device in accordance with the present invention;

FIG. 6 shows a menu of food selection of the operation in accordance with the present invention;

FIG. 7 shows a menu of unit systems in accordance with the present invention;

FIG. 8 shows a menu of nutrition/calorie calculation operation in accordance with the present invention; and

FIG. 9 shows a menu of disease list in accordance with the present invention.

### DETAILED DESCRIPTION OF THE PREFERRED EMBODIMENTS

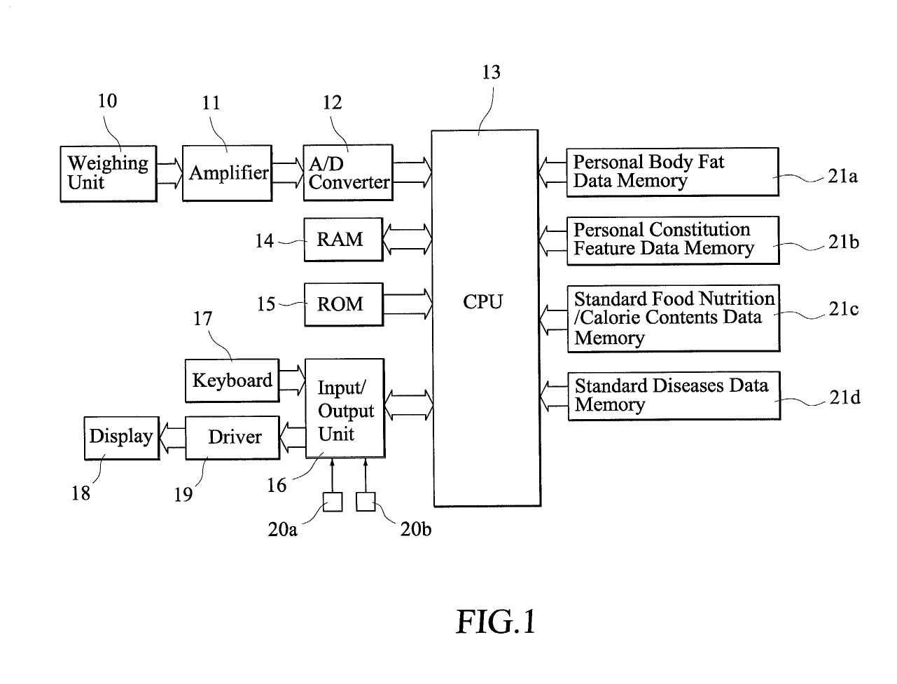

With reference to the drawings and in particular to FIG. 1, an electronic scale constructed in accordance with the present invention comprises a weighing unit 10 for weighing the weight of foods. The weighing unit 10 is capable of generating an analog signal indicating the weight being measured. An amplifier 11 is used to amplify the analog signal generated by the weighing unit 10. An analog-to-digital converter 12 is electrically coupled to the amplifier 11 for converting the amplified analog signal into a digital signal that is then applied to a processing unit 13, such as a microprocessor. A Random Access Memory 14 (RAM) is connected to the processing unit 13. A Read Only Memory 15 (ROM) such as an EEPROM is connected to the processing unit 15 for storing control program of the present invention.

Further, the processing unit 13 is provided with an input/output unit 16 to which a keyboard 17 is connected. A display 18 such as a LCD display or a LED matrix display is also connected to the input/output unit 16 via a driving device 19.

The electronic scale of the present invention is provided with a personal body fat data memory 21a, a personal constitution feature data memory 21b, a standard food nutrition/calorie contents data memory 21c, and a standard diseases data memory 21d.

A pair of electrodes 20a and 20b are also coupled to the input/output unit 16. The electrodes 20a and 20b are to be connected to the user for forming a current loop circuit. Based on signal generated by the current loop circuit of the electrodes 20a and 20b, the processing unit 13 determines the body fat of the user in accordance with known BIA analysis.

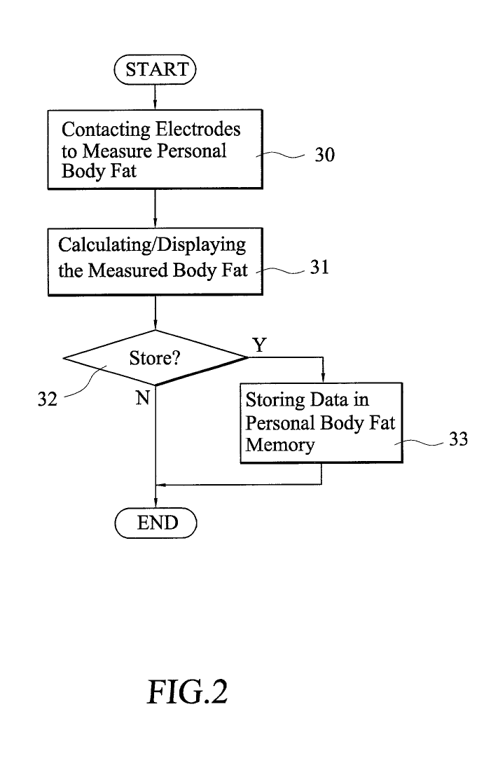

FIG. 2 shows a flow chart of the operation of the measuring device of the present invention for measuring body fat. To start the measurement, the user first contacts the electrodes 20 with his or her fingers to form the current loop circuit in step 30. The processing unit 13 calculates the body fat of the user, as well as fat percentage, in accordance with the BIA analysis in step 31, and the result may be shown on the display 18. In step 32, the user may select to store the measurement result and in step 33, the data are stored in the personal body fat data memory 21a.

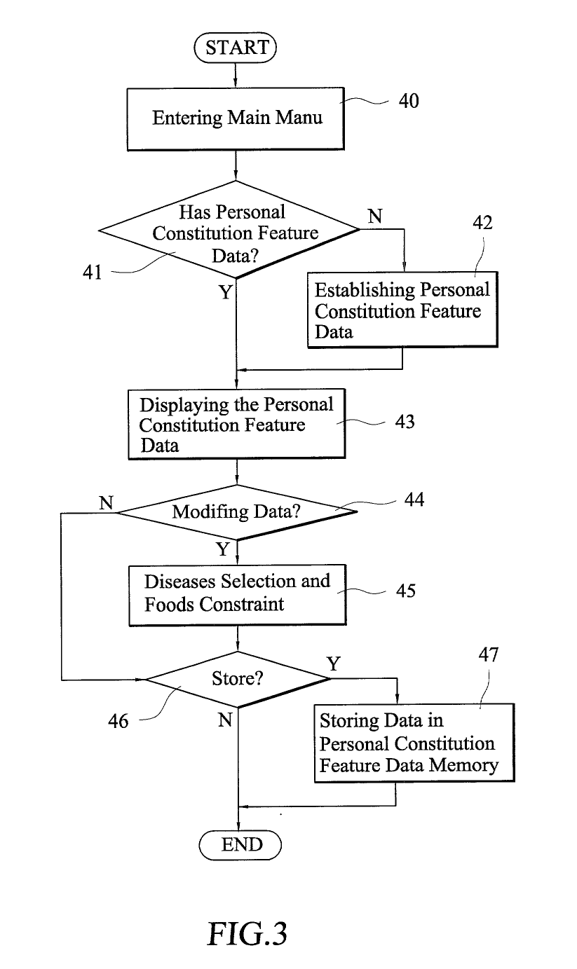

FIG. 3 shows a flow chart of establishing/modifying personal constitution feature data of the user. To start the operation, a main menu 100 is shown first in step 40. The main menu 100 can be retrieved by using the keyboard 17 and shown on the display 18. In step 41, the user may determine if his or her personal constitution feature data have already been established and stored. If the answer is negative, then the flow goes to step 42 for first time establishing the personal constitution feature data. If the personal constitution feature data have already been established, then the personal constitution feature data are shown on the display 18 in step 43. The personal constitution feature data may include personal body fat, fat percentage, diseases, constraints for food consumption or nutrition/calorie.

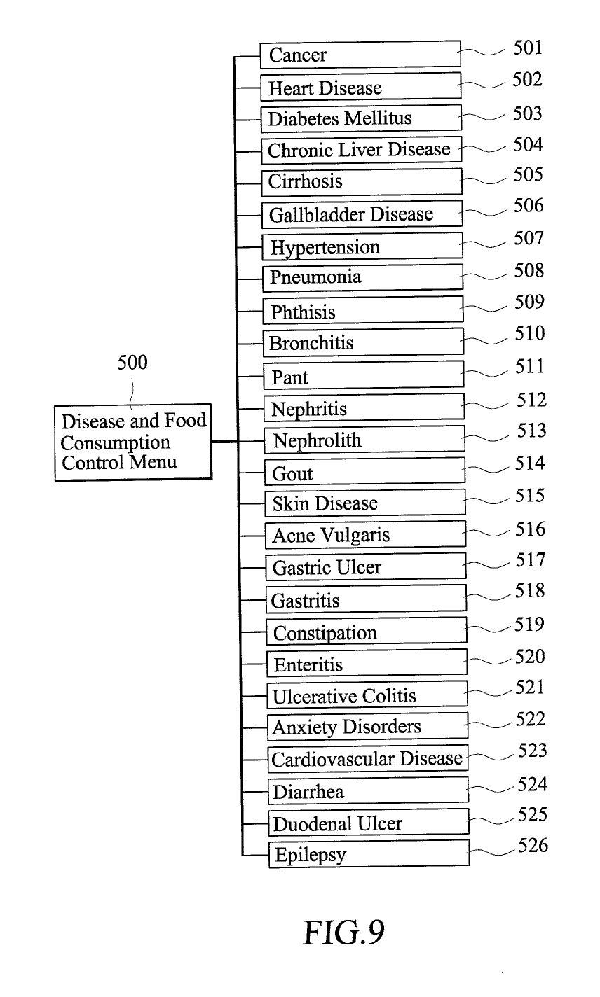

The user may then determine if the data are to be modified in step 44. If the answer is negative, then the operation ends. If the answer in step 44 is positive, the flow goes to step 45. In modifying the data in step 45, the user may enter new items or new constraints by means of the keyboard 17. These data may include body fat, fat percentage, personal diseases and constraints for food consumption. Preferably, a list of diseases is given for selection (with reference to FIG. 9). The list may include the following diseases, but not limited thereto: cancer, heart disease, diabetes mellitus, chronic liver disease, cirrhosis, gallbladder disease, hypertension, pneumonia, phthisis, bronchitis, pant, nephritis, nephrolith, gout, skin disease, acne vulgaris, gastric ulcer, gastritis, constipation, enteritis, ulcerative colitis, anxiety disorders, cardiovascular disease, diarrhea, duodenal ulcer and epilepsy.

The user may then select if to store the modified data in step 46. If yes, the data are stored in the personal constitution feature data memory 21b to update the old data in step 47.

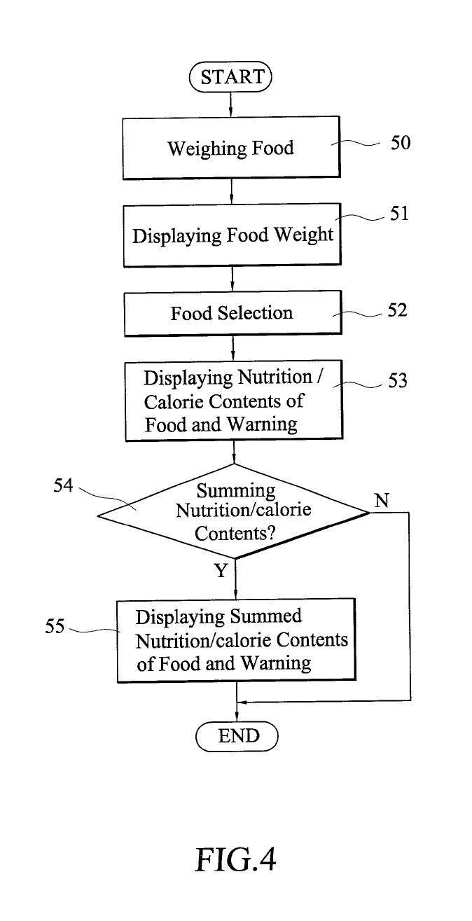

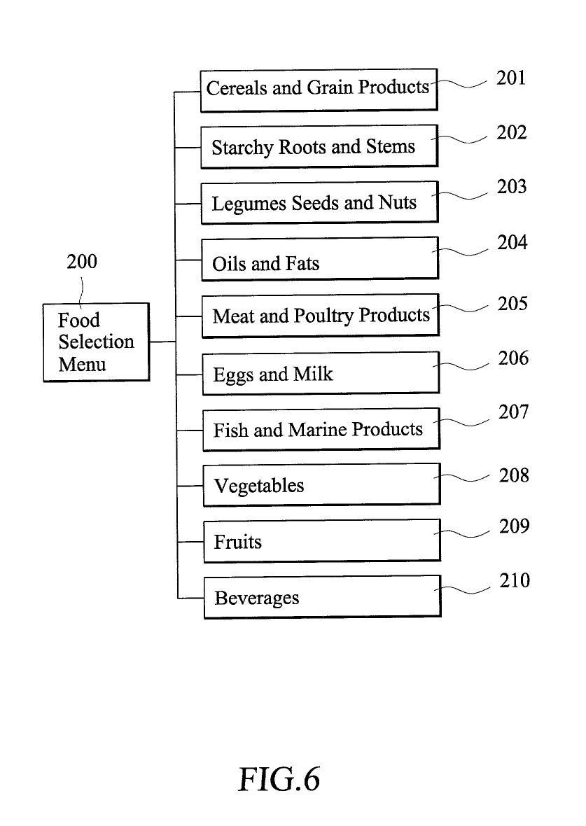

FIG. 4 shows a flow chart of measuring nutrition/calorie of food. The operation starts with weighing a selected food in step 50 by means of the weighing unit 10 shown in FIG. 1. The weight of the selected food is then displayed in step 51. A food list (FIG. 6) that is stored in the standard food nutrition/calorie contents data memory 21c is shown on the display 18 for user's selection of the category to which the food being measured belongs. The user may make use of the keyboard 17 to select among the categories of food in the food list. In a preferred embodiment of the present invention, food is classified in ten categories, including (1) cereals and grain products, (2) starchy roots and stems, (3) legumes seeds and nuts, (4) oils and fats, (5) meat and poultry products, (6) eggs and milk, (7) fish and marine products, (8) vegetables, (9) fruits, and (10) beverages.

In step 53, the processing unit 13 calculates the nutrition/calorie contents of the food being measured. The calculation is based on the data stored in the standard food nutrition/calorie contents data memory 21c associated with each category of the food list. The result of the calculation is shown on the display 18. The food nutrition/calorie calculated is also compared with the nutrition/calorie constraints of the user stored in the personal body fat data memory 21a and the personal constitution feature data memory 21b in order to determine if the consumption of the food is acceptable for healthy purposes. If the nutrition/calorie of the food being measured is improper to the user, then a visual warning signal may be shown on the display 18 or an audio warning signal may be generated by means of an audio device, such as a speaker.

If there is more than one categories of food to be measured, then in step 54 the user may select to add newly measured data to the previous data that has already been measured. This will be further discussed with reference to FIG. 8. In step 55, the data are summed together. Also, the summed nutrition/calorie contents of the food are shown on the display 18 in order to determine if the consumption of the summed food is acceptable for healthy purposes. If the nutrition/calorie of the food being measured is improper to the user, then a visual warning signal may be shown on the display 18 or an audio warning signal may be generated by means of an audio device, such as a speaker.

Preferably, a list is selectively provided on the display 18 for user's selection of a particular category of nutrition/calorie to be calculated. The list may include calorie, water, protein, fat, carbohydrate, fiber, ash, purine, cholesterol, calcium, phosphorus, iron, sodium, vitamins A, B1, B2, C, D, E, and nacin.

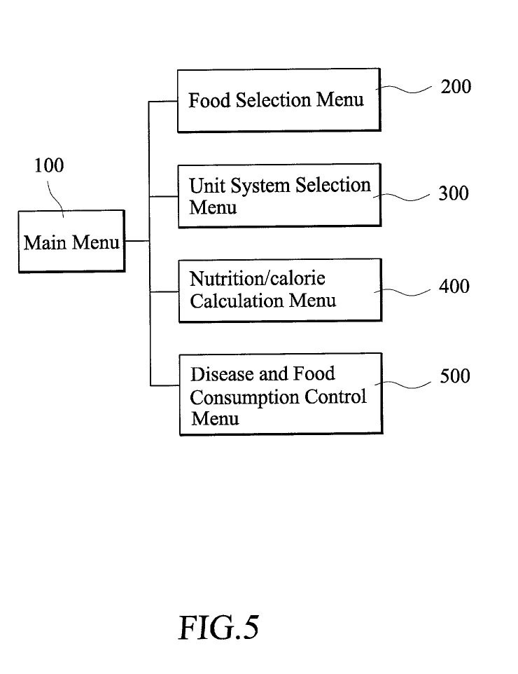

In accordance with a preferred embodiment of the present invention, a main menu 100 as shown in FIG. 5 may be provided and pre-stored in the standard food nutrition/calorie contents data memory 21c and the standard diseases data memory 21d. The main menu 100 may include four submenus, namely (1) food selection menu 200, (2) unit system selection menu 300, (3) nutrition/calorie calculation menu 400 and (4) disease and food consumption control menu 500.

As discussed previously and shown in FIG. 6, the food selection menu 200 comprises a list of ten categories of food, namely cereals and grain products 201, starchy roots and stems 202, legumes seeds and nuts 203, oils and fats 204, meat and poultry products 205, eggs and milk 206, fish and marine products 207, vegetables 208, fruits 209, and beverages 210.

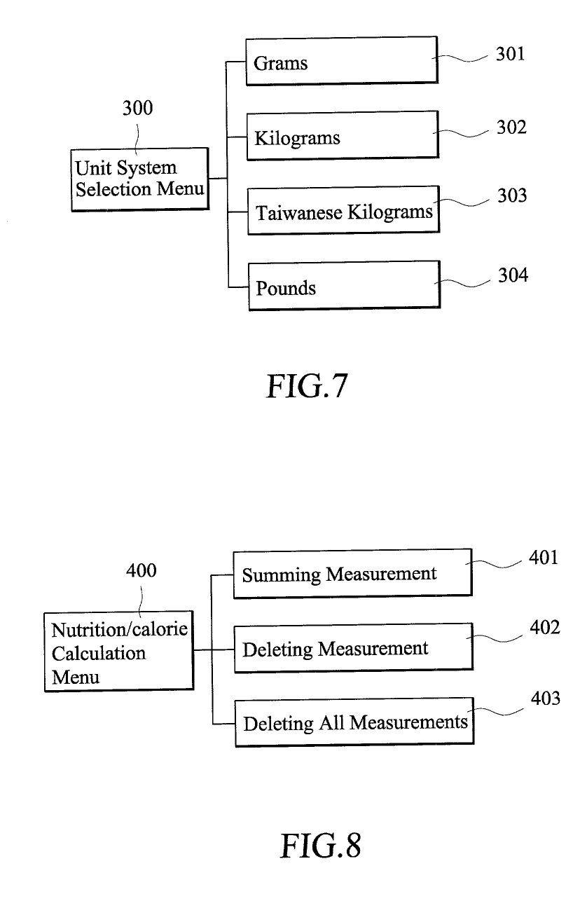

Referring to FIG. 7, the unit system selection menu 300 includes a list of different unit systems which allows a user to switch between different unit systems. In the embodiment illustrated, four units can be selected, including grams 301, kilograms 302, Taiwanese kilograms 303 and pounds 304. FIG. 8 shows the nutrition/calorie calculation menu 400 including three items respectively allowing a user to (a) add the current measurement to the previous measurement of the previous category of food (reference numeral 401), (b) delete the measurement of the previous category of food (reference numeral 402) and (c) delete all measurements (reference numeral 403).

FIG. 9 shows the disease and food consumption control menu 500 which may include the following diseases: cancer 501, heart disease 502, diabetes mellitus 503, chronic liver disease 504, cirrhosis 505, gallbladder disease 506, hypertension 507, pneumonia 508, phthisis 509, bronchitis 510, pant 511, nephritis 512, nephrolith 513, gout 514, skin disease 515, acne vulgaris 516, gastric ulcer 517, gastritis 518, constipation 519, enteritis 520, ulcerative colitis 521, anxiety disorders 522, cardiovascular disease 523, diarrhea 524, duodenal ulcer 525 and epilepsy 526. However, it is understood that the diseases are not limited to the above list. Other diseases can be added to the list and this can be done by user's modification of the contents of the standard diseases data memory 21d.

The disease and food consumption control menu 500 includes a number of diseases that are provided above. Each disease has its particular constraints to food consumption. Such information is pre-stored in the standard food nutrition/calorie contents data memory 21c and the standard diseases data memory 21d. Once a particular disease is selected and associated with the personal constitution feature data of the user stored in the personal constitution feature data memory 21b, the processing unit 13 may issue warnings to the user if the user is trying to consume improper amount/category of food.

Although the present invention has been described with reference to the preferred embodiments thereof, it is apparent to those skilled in the art that a variety of modifications and changes may be made without departing from the scope of the present invention which is intended to be defined by the appended claims.

We claim:

1. An electronic scale with a measuring device comprising:
a weighing unit for obtaining a weight of at least one selected food;
a processing unit for receiving the weight of the food;
a food nutrition/calorie contents data memory for storing food nutrition/calorie contents of a number of categories of foods;
a display connected to the processing; and
a user interface connected to the processing unit, allowing a user to access data stored in the memory;
wherein a plurality of categories of food are displayable on the display and accessible by the user through the user interface for selection of the category of a particular food being measured, the processing unit performing calculation to determine the nutrition/calorie contents of the selected food and displaying the calculation result on the display.

2. The electronic scale as claimed in claim 1, further comprising a personal constitution feature data memory coupled to the processing unit for storing a set of personal body constitution feature data therein.

3. The electronic scale as claimed in claim 2, the personal constitution feature data being associated with a list of personal diseases whereby a warning is issued by the processing unit if the calculation result indicates the particular food is not proper for the user.

4. The electronic scale as claimed in claim 1, further comprising a pair of electrodes to be attached to the user's body, the electrodes being coupled to the processing unit for forming a current loop circuit to determine the user's body fat.

5. The electronic scale as claimed in claim 4, further comprising a personal body fat data memory coupled to the processing unit for storing the body fat data.

6. The electronic scale as claimed in claim 1, wherein the food nutrition/calorie contents data memory comprises a list of categories of nutrition/calorie to be selected by a user, the list comprising calorie, water, protein, fat, carbohydrate, fiber, ash, purine, cholesterol, calcium, phosphorus, iron, sodium, vitamins A, B1, B2, C, D and E, and niacin.

7. The electronic scale as claimed in claim 1, further comprising a diseases data memory coupled to the processing unit for storing at least one standard disease data.

8. The electronic scale as claimed in claim 7, wherein the diseases data memory comprises a list of diseases and associated nutrition/calorie constraints, the list of diseases being accessible by a user through the user interface to select one or more of the diseases, the processing unit comparing the calculation result of the nutrition/calorie contents of the particular food to the nutrition/calorie constraints of the selected diseases and issuing a warning signal if the calculation result exceeds the constraints.

9. The electronic scale as claimed in claim 8, wherein the list of diseases comprises cancer, heart disease, diabetes mellitus, chronic liver disease, cirrhosis, gallbladder disease, hypertension, pneumonia, phthisis, bronchitis, pant, nephritis, nephrolith, gout, skin disease, acne vulgaris, gastric ulcer, gastritis, constipation, enteritis, ulcerative colitis, anxiety disorders, cardiovascular disease, diarrhea, duodenal ulcer, and epilepsy.
